(19) United States
(12) Patent Application Publication
(10) Pub. No.: US 2019/0142755 A1
(43) Pub. Date: May 16, 2019

(54) TABLET-IN-TABLET PHARMACEUTICAL COMPOSITION COMPRISING CYCLOPHOSPHAMIDE AND CAPECITABINE

(71) Applicant: INTAS PHARMACEUTICALS LTD., Ahmedabad (IN)

(72) Inventors: Priyank PATEL, Bodakdev Ahmedabad (IN); Mayur PATEL, Bodakdev Ahmedabad (IN); Mahendra PATEL, Bodakdev Ahmedabad (IN); Balvir SINGH, Bodakdev Ahmedabad (IN); Ashish SEHGAL, Bodakdev Ahmedabad (IN)

(73) Assignee: INTAS PHARMACEUTICALS LTD., Ahmedabad (IN)

(21) Appl. No.: 16/099,088

(22) PCT Filed: May 2, 2017
(86) PCT No.: PCT/IB2017/052534

§ 371 Date: Nov. 5, 2018

(30) Foreign Application Priority Data
May 3, 2016 (IN) 201621015342

(51) Int. Cl. A61K 9/28; A61K 9/24; A61K 9/20; A61K 31/7068; A61K 31/675

(52) U.S. Cl. CPC A61K 9/2893; A61K 9/209; A61K 31/675; A61K 31/7068; A61K 9/2095

(57) ABSTRACT

A stable tablet-in-tablet pharmaceutical composition having fixed dose combinations of cyclophosphamide and capecitabine and one or more pharmaceutically acceptable excipient. As disclosed is a process for the preparation thereof, and a method their use in treating cancer diseases.

### RELATED APPLICATIONS

This application is related to Indian Provisional Application 201621015342 filed on May 3, 2016 and is incorporated herein its entirely

### FIELD OF THE INVENTION

The present invention relates to a stable tablet-in-tablet pharmaceutical composition comprising fixed dose combinations of cyclophosphamide and capecitabine and one or more pharmaceutically acceptable excipient, process for the preparation thereof, and their use in treating cancer diseases.

### BACKGROUND OF THE INVENTION

Cyclophosphamide is an anti-cancer chemotherapy drug. This medication is classified as an alkylating agent. Cyclophosphamide is a prodrug, converted in the liver to active forms that slows down the growth of cancer cells. Cyclophosphamide requires enzymatic and chemical activation to produces active form. Cyclophosphamide is used alone or in combination with other drugs to treat various cancers like METASTATIC BREAST CANCER (MBC), ovarian cancer, leukemia. When administered orally, cyclophosphamide shows superior efficacy than when it is administered through intravenous route.

Among potential active cytotoxic drugs, capecitabine is the most commonly-used agent. Capecitabine has been approved by the Food and Drug Administration in the treatment of MBC patients resistant to anthracyclines and/or taxanes. Capecitabine widely used in different combination regimen due to better therapeutic & safety profile with lower side effects. In addition, the combination partner of capecitabine play important role for the activation of thymidine phosphorylase (TP) enzyme, which converts capecitabine to active 5-FU.

With increasing experience of capecitabine after its introduction, many clinicians found that oral administration of cyclophosphamide and capecitabine may have a greater potential for treatment of MBC due to anti-angiogenesis resulting from the metronomic dosage and upregulation of thymidine phosphorylase (dThdPase) by capecitabine.

In particular, a marked reduction in the tumor volume was seen during the time period coincident with the dThdPase up-regulation. In addition several clinical studies show that the efficacy of cyclophosphamide in combination with capecitabine was more than just an additive to synergistic effects.

Further interest in oral agents for the treatment of MBC has increased because many patients prefer oral to intravenous regimens due to administered at home, no need to hospitalization, fear of needles, and lower associated costs.

Inventors of the present invention investigated the development of pharmaceutical composition for oral administration. However, a stable pharmaceutical composition is tough to obtain due to incompatibility between the two therapeutic agents, specifically; the total impurity levels were found to have increased drastically. Cyclophosphamide is prone to undergo hydrolysis and easily degrade in presence of aqueous solution and light. Further cyclophosphamide is also labile to high temperatures. Hence it is very difficult to develop the stable pharmaceutical composition containing cyclophosphamide and one or more other active ingredients. The prior art references WO2015044961 and WO2015189807 disclose a bilayer oral tablet formulation to solve the above problems.

However, there is still an existing and continual need to provide a stable pharmaceutical composition of cyclophosphamide and capecitabine to allow convenient administration of both the drugs as a single tablet and to effective treatment with good acceptability at lower dose. To overcome the above issue, the inventors have provided herewith a novel stable tablet-in-tablet pharmaceutical composition comprising fixed dose combination of cyclophosphamide and capecitabine which meets the existing need.

### OBJECTS OF THE INVENTION

The object of the present invention is to provide a stable tablet-in-tablet pharmaceutical composition comprising a fixed dose combination of cyclophosphamide and capecitabine.

Another object of the present invention is to provide a stable tablet-in-tablet pharmaceutical composition comprising (a) a core comprising cyclophosphamide and one or more pharmaceutically acceptable excipient, and (b) an outer surrounding coat comprising capecitabine and one or more pharmaceutically acceptable excipient.

Another object of the present invention is to provide a stable tablet-in-tablet pharmaceutical composition comprising (a) a core comprising cyclophosphamide tablet, and (b) an outer surrounding coat comprising capecitabine granules.

Yet another object of the present invention is to provide a stable tablet-in-tablet pharmaceutical composition comprising (a) a core comprising cyclophosphamide tablet, and (b) an outer surrounding coat comprising capecitabine granules, wherein the core comprising cyclophosphamide tablet is optionally coated with a seal coat.

Yet another object of the present invention is to provide a stable tablet-in-tablet pharmaceutical composition comprising (a) a core comprising cyclophosphamide tablet, and (b) an outer surrounding coat comprising capecitabine granules, wherein the core comprising cyclophosphamide tablet is optionally coated with a seal coat, and further coated with one or more pharmaceutically acceptable excipients.

In yet another object of the invention is to provide a process for the preparation of a stable tablet-in-tablet pharmaceutical composition comprising (a) a core comprising cyclophosphamide and one or more pharmaceutically acceptable excipient, and (b) an outer surrounding coat comprising capecitabine and one or more pharmaceutically acceptable excipient.

### SUMMARY OF THE INVENTION

The present invention relates to a stable tablet-in-tablet pharmaceutical composition comprising a fixed dose combination of cyclophosphamide and capecitabine preferably in solid dosage form for oral administration, process for the preparation thereof, and their use in treating cancer diseases. Further the combination of capecitabine and cyclophosphamide is an effective, convenient and well-tolerated regimen for MBC.

### DETAILED DESCRIPTION

Cyclophosphamide and capecitabine are physically incompatible substances. Cyclophosphamide is easily degraded in presence of high temperature, moisture and light. Due to instability of the cyclophosphamide it is very difficult to develop a pharmaceutical composition comprising cyclophosphamide and capecitabine into a single dosage form.

In one object of the present invention, the pharmaceutical composition comprises a stable tablet-in-tablet pharmaceutical composition comprising (a) a core comprising cyclophosphamide and one or more pharmaceutically acceptable excipient, and (b) an outer surrounding coat comprising capecitabine and one or more pharmaceutically acceptable excipient.

Unless otherwise indicated, terms in this specification are intended to have their ordinary meaning in the relevant art.

The term “tablet-in-tablet” as used herein, refers to internal core of cyclophosphamide is covered by an outer coat of capecitabine that is compressed on to inner cyclophosphamide core.

The term “fixed dose combination” as used herein, refers to two active pharmaceutical ingredients cyclophosphamide and capecitabine combined in a single dosage form, which is manufactured and distributed in fixed doses.

The term “cyclophosphamide” as used herein, refers to 2-[bis(2-chloroethyl)amino]tetrahydro-2H-1,3,2-oxazaphosphorine2-oxide, chemically related to the nitrogen mustards.

The term “capecitabine” as used herein, refers 5′-deoxy-5-fluoro-N-[(pentyloxy) carbonyl]-cytidine.

The term “core” as used herein, refers to a single interior compartment of a unit dosage form comprising cyclophosphamide and one or more pharmaceutically acceptable excipient.

The term “outer surrounding coat” as used herein, refers to an exterior compartment of a unit dosage form comprising capecitabine and one or more pharmaceutically acceptable excipient, which completely surrounds the core of cyclophosphamide.

The term “stable” as used herein, refers to a composition in which the active pharmaceutical ingredients (i.e., cyclophosphamide and capecitabine) are present in an amount of at least 90%, and preferably at least 95%, or at least 99.5% of the originally specified amount for each such ingredient. In certain embodiments, the term “stable” refer to a compound wherein the cyclophosphamide related impurities A, B, C, D, unspecified and total impurities are individually not more than 0.06% w/w, 0.2% w/w, 0.06% w/w, 0.3% w/w, 0.4% w/w and 0.5% w/w respectively, and capecitabine related impurities A, B, C. unspecified and total impurity are individually not more than 0.4% w/w, 0.08% w/w, 0.06% w/w, 0.03% w/w and 0.5% respectively when stored for 6 month at 2-8° C., 25° C./60% Relative Humidity (RH) and 30° C./75% RH. In a particular embodiment, impurities of cyclophosphamide and capecitabine defined as below:

| | Chemical name |
|---|---|
| Cyclophosphamide Impurities | |
| Impurity A | Bis (2-chloroethyl)amine hydrochloride |
| Impurity B | 3-(2-chloroethyl)-2-oxo-2-hydroxy-1,3,6,2 |
| | oxadiazaphosphonane |
| Impurity C | 3-aminopropyl dihydrogen phosphate. |
| Impurity D | 3-[3-(2-chloroethylamino) ethyl amino] |
| | propyl dihydrogen phosphate |
| Capecitabine Impurities | |
| Impurity A | 5′-Deoxy-5-fluorocytidine |
| Impurity B | 5′-Deoxy-5-fluorouridine |
| Impurity C | 2′,3′-O-Carbonyl-5′-deoxy-5-fluoro-N4- |
| | (pentyloxycarbonyl)cytidine |

In one object, the present invention relates to a novel stable tablet-in-tablet pharmaceutical composition comprising a fixed dose combination of cyclophosphamide and capecitabine in a single dosage form for oral administration and used in effective and well-tolerated regimen for MBC.

According to one of the embodiments, the core portion comprises 10-100 mg, preferably 20-80 mg, more preferably 20-60 mg of cyclophosphamide and outer surrounding coat comprises 50-1800 mg, preferably 100-1500 mg, more preferably 300-800 mg of capecitabine.

In one preferred embodiment, the cyclophosphamide is present in about 30 mg and the capecitabine is present in about 700 mg.

In another preferred embodiment, the cyclophosphamide is present in about 20 mg and the capecitabine is present in about 400 mg.

According to the invention the fixed dose combination composition of cyclophosphamide and capecitabine is preferable used as solid formulation, for example in the form of tablets, more preferably tablet-in-tablet.

In one of the embodiments, the core portion of cyclophosphamide present is in the form of tablets and comprises cyclophosphamide and one or more pharmaceutically acceptable excipients.

Further in one of the other embodiment, the cyclophosphamide tablet is optionally coated with a seal coat comprising a coating solution containing one or more pharmaceutically acceptable excipients.

Further in one of the embodiment, the outer surrounding coat of capecitabine is present in the form of granules comprising capecitabine and one or more pharmaceutically acceptable excipient.

In one preferred embodiment, the stable tablet-in-tablet pharmaceutically composition comprises:
(a) core in the form of tablets comprising cyclophosphamide and one or more pharmaceutically acceptable excipient; and
(b) an outer surrounding coat in the form of granules comprising capecitabine and one or more pharmaceutically acceptable excipient.

In one of the embodiment, the core in the form of tablets comprising cyclophosphamide is optionally coated with a seal coat which acts as a protective layer. The said seal coat is a non-functional coating and is abundantly known to a person skilled in the art

The stable composition of the present invention may include one or more pharmaceutically acceptable excipients to enable formation of a present composition. The pharmaceutical excipients includes, but not limited to one or more diluents, binders, disintegrants, glidants, lubricants, plasticizer, opacifying agent, and colorants. The amount of each excipient in a solid dosage formulation may vary within ranges conventional in the art.

Examples of suitable diluents include, but not limited to microcrystalline cellulose, microfine cellulose, lactose, starch, pregelatinized starch, calcium carbonate, calcium sulfate, sugar or sugar derivatives, dibasic calcium phosphate dihydrate, tribasic calcium phosphate, mannitol, powdered cellulose and sorbitol.

Examples of suitable binders include, but not limited to povidone, hydroxypropyl methyl cellulose, hydroxypropyl cellulose, microcrystalline cellulose, acacia, starch, pregelatinized starch, carboxymethylcellulose sodium, ethyl cellulose and gelatin.

Examples of suitable disintegrants include, but not limited to croscarmellose sodium, crospovidone, sodium starch glycolate, microcrystalline cellulose, polacrilin potassium, pregelatinized starch, carboxymethylcellulose calcium and starch.

Examples of suitable glidants include, but not limited to colloidal silicon dioxide, magnesium trisilicate, powdered cellulose, starch and talc.

Examples of suitable lubricants include, but not limited to magnesium stearate, calcium stearate, glyceryl monostearate, glyceryl palmitostearate, hydrogenated castor oil, hydrogenated vegetable oil, mineral oil, polyethylene glycol, sodium lauryl sulfate, sodium stearyl fumarate, stearic acid, talc, and zinc stearate.

In yet another object of the invention is to provide a process for the preparation of a stable tablet-in-tablet pharmaceutical composition comprising (a) a core comprising cyclophosphamide and one or more pharmaceutically acceptable excipient, and (b) an outer surrounding coat comprising capecitabine and one or more pharmaceutically acceptable excipient.

In one embodiment, a tablet-in-tablet dosage form of cyclophosphamide and capecitabine may be prepared by compressing the granules of capecitabine with cyclophosphamide tablet along with one or more pharmaceutically acceptable excipient in such a way that inner optionally seal coated tablet of cyclophosphamide tablet is coated with an outer surrounding coat of capecitabine granules using a suitable tablet press.

The pharmaceutical composition described herein may be prepared by conventional technology well known to those skilled in the art such as wet granulation, dry granulation and direct compression and the like.

More preferably in the present invention, the core portion comprising cyclophosphamide and one or more pharmaceutically acceptable excipient were prepared by blending the excipients for direct compression whilst the outer surrounding coat comprising cyclophosphamide granules were prepared by wet granulation as hereinafter described.

In one of the embodiment, core portion comprising cyclophosphamide and one or more pharmaceutically acceptable excipient is prepared by direct compression comprising following steps:
(a) mixing the cyclophosphamide with one or more diluent, one or more binder and one or more disintegrant in suitable mixer;
(b) lubricating the mixture obtained in step (a) by blending with a suitable sifted lubricant;
(c) compressing the lubricating mixture obtained in step (b) into a tablet; and
(d) optionally coating the above compressed tablet to prepare the inner core portion as cyclophosphamide tablet

In one preferred embodiment, the inner core tablet comprising cyclophosphamide having d90 particle size less than 100 microns.

In one of the embodiment, core portion comprising cyclophosphamide tablets containing lactose and microcrystalline cellulose as a preferred diluent, sodium starch glycolate as a preferred disintegrant, acacia as a preferred binder and magnesium stearate as a preferred lubricant.

In one of other embodiment, cyclophosphamide tablets optionally coated with a seal coat wherein the coating solution comprising mixture of polymers, plasticizers, film formers, opacifying agents, coloring agents and other excipients. The coating solution is formulated in purified water or any suitable organic solvent can be selected from ethanol, isopropyl alcohol and methylene chloride. The organic solvent is a volatile component, which does not remain in final composition.

In a preferred embodiment, coating solution comprising hydroxypropyl methyl cellulose and hydroxypropyl cellulose as a film former, talc as a glidant, titanium dioxide as opacifier, polyethylene glycol as a plasticizer, iron oxide red & iron oxide yellow as a coloring agents which are prepared in isopropyl alcohol and dichloro methane.

In one of the other embodiment, the outer surrounding coat comprising capecitabine and one or more pharmaceutically acceptable excipient is prepared by wet granulation method comprising following steps:
(a) sifting of capecitabine, one or more diluent, a portion of disintegrant through appropriate mesh and mixing in a suitable mixer;
(b) dissolving a binder in a solvent to produce a granulation liquid;
(c) granulating the dry mixture obtained in step (a) using the granulation liquid of step (b);
(d) drying the wet granules obtained in step (c) and optionally screening the dried granules;
(e) blending the dried granules obtained in step (d) with remaining portion of disintegrant and sifted glidant; and
(f) lubricating the blended granules obtained in step (e) with sifted lubricant to prepare the outer surrounding coat.

In certain embodiments, the outer surrounding coat of capecitabine granules comprising lactose and microcrystalline cellulose as a preferred diluent, croscarmellose sodium as a preferred disintegrant, hydroxypropyl methylcellulose as a preferred binder, colloidal silicon dioxide as a preferred glidant and magnesium stearate used as a preferred lubricant.

In the wet granulation process the granulating liquid is a solvent such as purified water, ethanol, isopropanol, acetone or mixture thereof, preferably purified water used as a solvent for granulation liquid.

In one of the embodiment, final tablet-in-tablet composition is prepared by the compressing the inner core of cyclophosphamide tablets and outer surrounding coat of capecitabine granules in a suitable tablet press. In one of another embodiment, the tablet-in-tablet is further coated with a coating solution. The details of coating solution are described herein above.

In preferred embodiment, a stable tablet-in-tablet pharmaceutical composition comprises cyclophosphamide and capecitabine wherein the inner tablet of cyclophosphamide are fed into the tablet-in-tablet press with capecitabine granules and the press operated again such that a final pharmaceutical composition is of cyclophosphamide tablet inside capecitabine granules.

The cyclophosphamide seal coated tablets and capecitabine granules can be optionally filed into suitable capsule shells using techniques of partial filing method and tablet-in-capsule.

Stability study of present invention comprising cyclophosphamide and capecitabine was carried out for a period of 6 month at various stability condition of 2-8° C., 25° C./60% Relative Humidity (RH) and 30° C./75% RH and were found to be stable.

Further the present invention provides a pharmaceutical composition comprising a stable tablet-in-tablet pharmaceutical composition comprising a fixed dose combinations of cyclophosphamide and capecitabine and one or more pharmaceutically acceptable excipients thereof have a greater potential for treatment of MBC.

In addition, the present invention provides a better therapeutic efficacy by combined administered of capecitabine and cyclophosphamide rather than when used separately.

### EXAMPLES

The present invention has been described by way of example only. It is to be recognized that modifications falling within the scope and spirit of the claims, which would be obvious to a person skilled in the art based upon the disclosure herein, are also considered to be included within the scope of this invention. The scope of the invention is in no manner limited by the disclosed example.

### Reference Example 1

| | | |
|---|---|---|
| Sr. No. | Ingredients | Qty./Tablet (% w/w) |

Cyclophosphamide Tablet

| 1 | Cyclophosphamide | 2-3% |
|---|---|---|
| 2 | Diluent | 3-5% |
| 3 | Disintegrant | 1-5% |
| 4 | Binder | 1-5% |
| 5 | Lubricant | 0.1-2% |
| Core weight of cyclophosphamide | | 7-10%% |
| 6 | Coating agents | 0.1-2% |
| 7 | Solvent | q.s. |

| | |
|---|---|
| Coated weight of cyclophosphamide | 8-10% |

Capecitabine granules

| 8 | Capecitabine | 60-70% |
|---|---|---|
| 9 | Diluent | 5-15% |
| 10 | Disintegrant | 3-10% |
| 11 | Binder | 2-5% |
| 12 | Purified Water | q.s. |
| 13 | Glidant | 0.1-1% |
| 14 | Lubricant | 1-2% |
| Total of Capecitabine granules | | 85-90% |
| 15 | Cyclophosphamide tablets | 8-10% |
| 16 | Capecitabine granules | 85-90% |
| Core tablet weight | | 95-98% |
| 17 | Coating agents | 2-4% |
| 18 | Purified water | q.s. |

| | |
|---|---|
| Film coated tablet weight | 100% |

Manufacturing Process:

Preparation of Cyclophosphamide Tablet:
1. mixing the cyclophosphamide with diluent, binder and disintegrant in suitable mixer.
2. lubricating the mixture obtained in step (1) by blending with a suitable sifted lubricant.
3. compressing the lubricating mixture obtained in step (b) into a tablet.
4. coating the above compressed tablet to prepare the inner core portion as cyclophosphamide tablet.

Preparation of Capecitabine Granules:
5. sifting of capecitabine, one or more diluent, a portion of disintegrant through appropriate mesh and mixing in a suitable mixer.
6. dissolving a binder in a solvent to produce a granulation liquid.
7. granulating the dry mixture obtained in step (5) using the granulation liquid of step (6).
8. drying the wet granules obtained in step (7) and optionally screening the dried granules.
9. blending the dried granules obtained in step (8) with remaining portion of disintegrant and sifted glidant.
10. lubricating the blended granules obtained in step (9) with sifted lubricant to prepare the outer surrounding coat of capecitabine granules.

Tablet in Tablet Compression & Film Coating of Tablet in Tablet
11. Compress cyclophosphamide tablets of step 4 and granules of capecitabine of step 10 using rotary tablet compression machine.
12. Coat the tablet obtained in step (11) with a coating solution.
13. Pack the film coated tablets in suitable pack using packaging machine.

### Example 1

| | | |
|---|---|---|
| Sr. No. | Ingredients | Qty./Tablet |

Cyclophosphamide Tablet

| 1 | Cyclophosphamide | 30.00 |
|---|---|---|
| 2 | Lactose Monohydrate | 18.00 |
| 3 | Microcrystalline cellulose | 31.20 |
| 4 | Sodium starch glycolate | 5.40 |
| 5 | Acacia | 3.60 |
| 6 | Magnesium stearate | 1.80 |
| Core weight of cyclophosphamide | | 90.00 |
| 7 | Opadry | 9.00 |
| 8 | Isopropyl alcohol | q.s. |
| 9 | Dichloromethane | q.s. |

| | |
|---|---|
| Coated weight of cyclophosphamide | 99.00 |

Capecitabine granules

| 10 | Capecitabine | 700.00 |
|---|---|---|
| 11 | Microcrystalline cellulose | 58.22 |
| 12 | Lactose Anhydrous | 67.25 |
| 13 | Croscarmellose sodium | 46.38 |
| 14 | Hydroxypropyl methylcellulose | 32.46 |
| 15 | Purified water | q.s. |
| 16 | Colloidal silicon dioxide | 4.64 |
| 17 | Magnesium Stearate | 18.55 |
| Total of Capecitabine granules | | 927.50 |
| 18 | Cyclophosphamide tablets | 99.00 |
| 19 | Capecitabine granules | 927.50 |
| Core tablet weight | | 1026.50 |
| 20 | Opadry | 30.80 |
| 21 | Purified water | q.s. |

| | |
|---|---|
| Film coated tablet weight | 1057.30 |

Manufacturing Process:

Preparation of Cyclophosphamide Tablet
1. mixing the cyclophosphamide, microcrystalline cellulose, lactose monohydrate, sodium starch glycolate and acacia in a suitable mixer.
2. lubricating the mixture obtained in step (1) by blending with magnesium stearate.
3. compressing the lubricating mixture obtained in step (b) into a tablet.
4. coating the above compressed tablet using opadry in purified water to prepare the inner core portion as cyclophosphamide tablet.

Preparation of Capecitabine Granules:
5. sifting of capecitabine, microcrystalline cellulose, lactose anhydrous, and croscarmellose sodium through appropriate mesh and mixing in a suitable mixer.
6. dissolving hydroxypropyl methylcellulose in a solvent to produce a granulation liquid.
7. granulating the dry mixture obtained in step (5) using the granulation liquid of step (6).
8. drying the wet granules obtained in step (7) and optionally screening the dried granules.
9. blending the dried granules obtained in step (8) with remaining portion of croscarmellose sodium and colloidal silicon dioxide.
10. lubricating the blended granules obtained in step (9) with magnesium stearate to prepare the outer surrounding coat of capecitabine granules.

Tablet in Tablet Compression & Film Coating of Tablet in Tablet
11. Compress cyclophosphamide tablets of step 4 and granules of capecitabine of step 10 using rotary tablet compression machine.
12. Coat the tablet obtained in step (11) with opadry in purified water.
13. Pack the film coated tablets in suitable pack using packaging machine.

In-Vitro Dissolution Profile & Stability Study

The tablet-in-tablet fixed dose combination of cyclophosphamide and capecitabine prepared as per the present invention were subjected to dissolution studies in 900 ml of phosphate buffer pH 6.8 at 37±0.5° C. using basket apparatus with rotational speed at 100 rpm.

Table 1 provides dissolution profile of tablets prepared according to Example 1

| | | | | 25° C./ | | 30° C./ | |
|---|---|---|---|---|---|---|---|
| | | 2-8° C. | | 60% RH | | 75% RH | |
| Dissolution Study | Initial | 1 M | 6 M | 1 M | 6 M | 1 M | 6 M |
| Cyclophosphamide | 104 | 107 | 109 | 103 | 103 | 105 | 106 |
| Capecitabine | 100 | 98 | 100 | 99 | 102 | 97 | 96 |

The above dissolution study data comply with the dissolution testing requirements of immediate release solid oral dosage forms.

Stability Study

(i) Impurity Profile

The study of cyclophosphamide and capecitabine impurities were carried out at a temperature range of 2-8° C., or at temperature 25° C. (60% RH), or at 30° C. (75% RH) are suitable at least 6 month. The impurity profile results obtained are as below:

| | | | | | 25° C./ | | 30° C./ | |
|---|---|---|---|---|---|---|---|---|
| | | | 2-8° C. | | 60% RH | | 75% RH | |
| | Limits | Stability Initial | 1 M | 6 M | 1 M | 6 M | 1 M | 6 M |
| Related substances | | | | | | | | |
| Cyclophosphamide | | | | | | | | |
| Impurity A | NMT 1.5 | ND | ND | ND | ND | ND | ND | ND |
| Impurity B | NMT 3.5 | 0.116 | 0.154 | 0.197 | 0.195 | 0.106 | 0.196 | 0.07 |
| Impurity C | NMT 1.5 | ND | ND | ND | ND | ND | ND | ND |
| Impurity D | NMT 2.0 | ND | ND | ND | 0.077 | 0.151 | 0.064 | 0.243 |
| Individual | NMT 1.0 | ND | ND | 0.066 | 0.081 | 0.129 | 0.112 | 0.333 |
| unspecified | | | | | | | | |
| impurity | | | | | | | | |
| Total Impurities | NMT 6.0 | 0.116 | 0.154 | 0.263 | 0.425 | 0.599 | 0.461 | 0.972 |
| Capecitabine | | | | | | | | |
| Impurity A | NMT 1.0 | 0.201 | 0.2 | 0.219 | 0.219 | 0.281 | 0.237 | 0.348 |
| Impurity B | NMT 1.0 | 0.033 | 0.043 | 0.04 | 0.048 | 0.056 | 0.052 | 0.072 |
| Impurity C | NMT 0.5 | 0.051 | 0.049 | 0.049 | 0.048 | 0.051 | 0.05 | 0.049 |
| Individual | NMT 0.1 | 0.021 | 0.017 | 0.016 | 0.014 | 0.012 | BQL | 0.012 |
| unspecified | | | | | | | | |
| impurity | | | | | | | | |
| Total Impurities | NMT 0.5 | 0.306 | 0.309 | 0.324 | 0.329 | 0.4 | 0.339 | 0.399 |

ND: Not Detected,
BQL: Below Quantification limit.

Impurity profile of the pharmaceutical compositions of example 1 meets the acceptance criteria of individual and total impurities of cyclophosphamide and capecitabine as disclosed hereinabove.

(ii) Assay:

| | | | | | 25° C./ | | 30° C./ | |
|---|---|---|---|---|---|---|---|---|
| | Stability | | 2-8° C. | | 60% RH | | 75% RH | |
| Assay | Limits | Initial | 1 M | 6 M | 1 M | 6 M | 1 M | 6 M |
| Cyclophosphamide | 90%-110% | 108 | 107.2 | 105.7 | 107.4 | 102.8 | 107.8 | 102.8 |
| Capecitabine | 92.5%-107.5% | 98.7 | 99.8 | 98.8 | 98.9 | 98.8 | 99.2 | 98.2 |

As is evident from the above data, the assay of cyclophosphamide and capecitabine are within the acceptable limits of 90%-110% and 92.5%-107.5% respectively, when stored at a temperature range of 2-8° C., 25° C. (60% RH), and 30° C. (75% RH) for up to 6 month.

1. A stable tablet-in-tablet pharmaceutical composition comprising a fixed dose combination of cyclophosphamide and capecitabine.

2. The stable tablet-in-tablet pharmaceutical composition according to claim 1, comprises (a) a core comprising cyclophosphamide and one or more pharmaceutically acceptable excipient, and (b) an outer surrounding coat comprising capecitabine and one or more pharmaceutically acceptable excipient.

3. The stable tablet-in-tablet pharmaceutical composition according to claim 2, wherein the core comprising 20 to 60 mg of cyclophosphamide.

4. The stable tablet-in-tablet pharmaceutical composition according to claim 2, wherein the outer coat comprising 50 to 1800 mg of capecitabine.

5. The stable tablet-in-tablet pharmaceutical composition according to claim 2, wherein the core comprising cyclophosphamide having d90 particle size less than 100 microns.

6. The stable tablet-in-tablet pharmaceutical composition according to claim 2, wherein the core comprising cyclophosphamide tablet is optionally coated with a seal coat.

7. The stable tablet-in-tablet pharmaceutical composition according to claim 2, wherein the pharmaceutically acceptable excipients can be selected form one or more diluents, one or more binders, one or more disintegrants, one or more glidants, one or more lubricants, one or more plasticizer, one or more opacifying agent, one or more colorants and combinations thereof.

8. The stable tablet-in-tablet pharmaceutical composition according to claim 2, wherein the inner core comprising cyclophosphamide is prepared by direct compression comprising following steps:
(a) mixing the cyclophosphamide with one or more diluent, one or more binder and one or more disintegrant in suitable mixer;
(b) lubricating the mixture obtained in step (a) by blending with a suitable sifted lubricant;
(c) compressing the lubricating mixture obtained in step (b) into a tablet; and
(d) optionally coating the above compressed tablet to prepare the inner core portion as cyclophosphamide tablet.

9. The stable tablet-in-tablet pharmaceutical composition according to claim 2, wherein the outer coat comprising capecitabine granules is prepared by wet granulation method comprising following steps:
(a) sifting of capecitabine, one or more diluent, a portion of disintegrant through appropriate mesh and mixing in a suitable mixer;
(b) dissolving a binder in a solvent to produce a granulation liquid;
(c) granulating the dry mixture obtained in step (a) using the granulation liquid of step (b);
(d) drying the wet granules obtained in step (c) and optionally screening the dried granules;
(e) blending the dried granules obtained in step (d) with remaining portion of disintegrant and sifted glidant; and
(f) lubricating the blended granules obtained in step (e) with sifted lubricant to prepare the outer surrounding coat.

10. The stable tablet-in-tablet pharmaceutical composition according to claim 2, wherein the said composition comprising at least 90% of the cyclophosphamide and capecitabine when stored for 6 month at 2-8° C., 25° C./60% RH and 30° C./75% RH.
